(19) United States
(12) Patent Application Publication
(10) Pub. No.: US 2025/0023261 A1
(43) Pub. Date: Jan. 16, 2025

(54) ELECTRIC WIRE CONNECTION STRUCTURE AND ELECTRIC WIRE CONNECTION STRUCTURE MANUFACTURING METHOD

(71) Applicant: Proterial, Ltd., Tokyo (JP)

(72) Inventor: Kenichi TAMURA, Tokyo (JP)

(73) Assignee: Proterial, Ltd., Tokyo (JP)

(21) Appl. No.: 18/771,560

(22) Filed: Jul. 12, 2024

(30) Foreign Application Priority Data
Jul. 14, 2023 (JP) 2023-115887

(51) Int. Cl. H01R 12/53; A61M 25/09

(52) U.S. Cl. CPC H01R 12/53; A61M 25/09; H01R 2201/12

(57) ABSTRACT

An electric wire connection structure includes a plurality of electric wires each having a covered portion, at which a core wire is covered with a covering, and a core wire exposed portion, at which the core wire is exposed from the covering, the exposed core wire portions being arranged in parallel to each other; an electronic component including a plurality of electrodes respectively connected to the core wire exposed portions of the plurality of electric wires; and a core wire fixing member to fix the plurality of core wire exposed portions to each other.

### CROSS-REFERENCE TO RELATED APPLICATIONS

The present application is based on Japanese patent application No. 2023-115887 filed on Jul. 14, 2023, the entire contents of which are incorporated herein by reference.

### TECHNICAL FIELD

This disclosure relates to an electric wire connection structure and a method for manufacturing an electric wire connection structure.

### BACKGROUND OF THE INVENTION

Patent Literature 1 discloses an electric wire connection structure that includes plural electrodes provided on a pressure sensor for catheter treatment and plural electric wires respectively connected to the plural electrodes. The plural electric wires are arranged so that core wire exposed portions, at which core wires are exposed, are aligned in parallel and connected to the plural electrodes.

### CITATION LIST

Patent Literature 1: US Patent Application Publication No. 2015/0137274

### SUMMARY OF THE INVENTION

In the electric wire connection structure described in Patent Literature 1, it is difficult to maintain the distances between the plural core wire exposed portions.

The invention was made in view of such circumstances and it is an object to provide an electric wire connection structure in which the distances between plural core wire exposed portions are easily maintained, and a method for manufacturing an electric wire connection structure.

To achieve the above object, one aspect of the invention provides an electric wire connection structure, comprising:
a plurality of electric wires each comprising a covered portion, at which a core wire is covered with a covering, and a core wire exposed portion, at which the core wire is exposed from the covering, the exposed core wire portions being arranged in parallel to each other;
an electronic component comprising a plurality of electrodes respectively connected to the core wire exposed portions of the plurality of electric wires; and
a core wire fixing member to fix the plurality of core wire exposed portions to each other.

To achieve the above object, another aspect of the invention provides a method for manufacturing an electric wire connection structure, comprising:
arranging in parallel core wire exposed portions of a plurality of electric wires at which core wires are exposed from coverings, and fixing the core wire exposed portions with a core wire fixing member, thereby fixing electric wires; and
after the fixing electric wires, connecting the core wire exposed portions of the plurality of electric wires respectively to a plurality of electrodes of an electronic component.

### Advantageous Effects of the Invention

According to the invention, it is possible to provide an electric wire connection structure in which the distances between plural core wire exposed portions are easily maintained and a method for manufacturing an electric wire connection structure.

### BRIEF DESCRIPTION OF DRAWINGS

FIG. 1 is a perspective view showing an electric wire connection structure in the first embodiment.

FIG. 2 is a plan view showing the electric wire connection structure in the first embodiment.

FIG. 3 is a cross-sectional view passing through core wires of plural electric wires and showing the electric wire connection structure in the first embodiment.

FIG. 4 is a cross-sectional view taken along line IV-IV in FIG. 2 as viewed in the arrow direction.

FIG. 5 is a cross-sectional view taken along line V-V in FIG. 2 as viewed in the arrow direction.

FIG. 6 is a plan view showing three electric wires prepared in an electric wire preparation step in the first embodiment.

FIG. 7 is a plan view showing the three electric wires and a core wire fixing member after an electric wire fixing step in the first embodiment.

FIG. 8 is a plan view showing an electronic component, three conductive bonding materials, the three electric wires, and the core wire fixing member after a connecting step in the first embodiment.

FIG. 9 is a cross-sectional view showing the electric wire connection structure in the second embodiment.

### DETAILED DESCRIPTION OF THE INVENTION

### First Embodiment

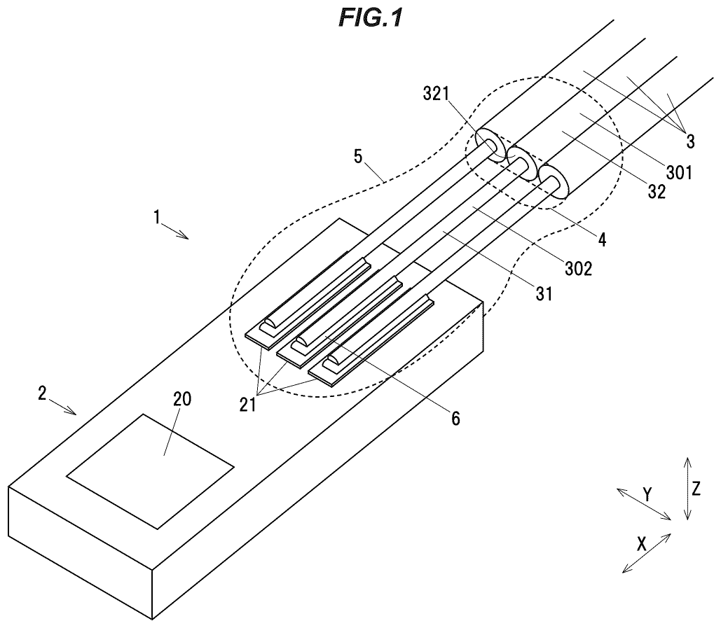

The first embodiment will be described in reference to FIGS. 1 to 8. The embodiment below is described as a preferred illustrative example for implementing the invention. Although some part of the embodiment specifically illustrates various technically preferable matters, the technical scope of the invention is not limited to such specific aspects.

### (Electric Wire Connection Structure)

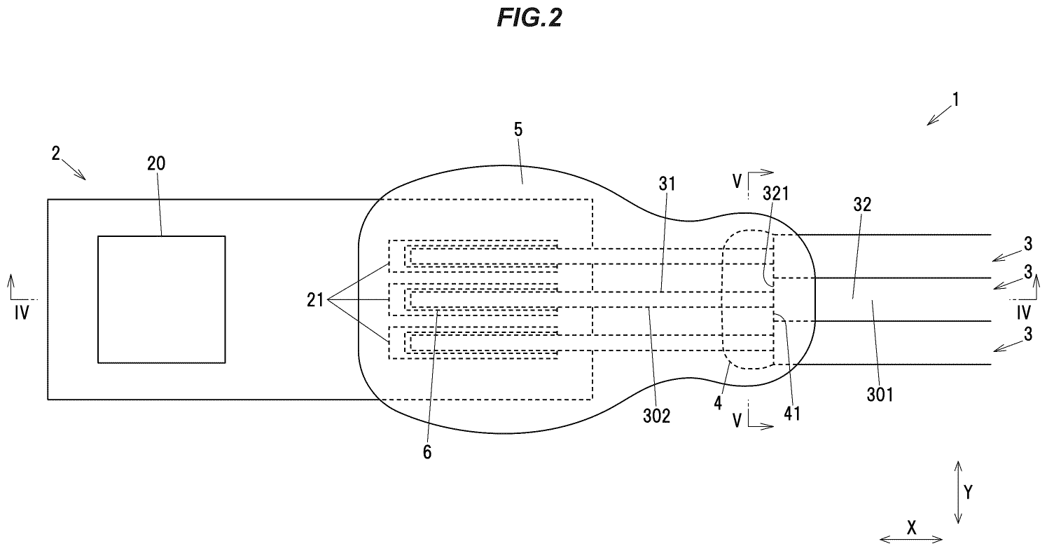

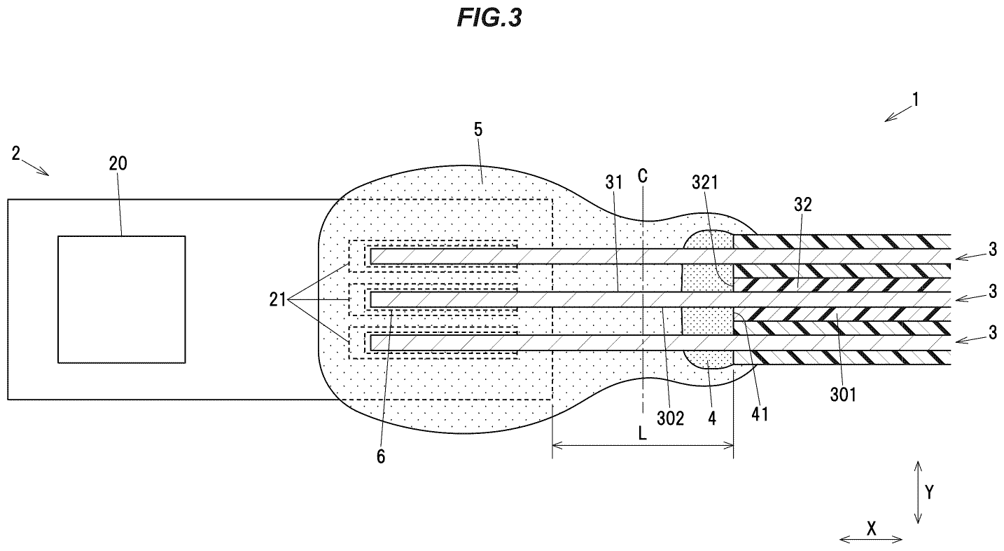

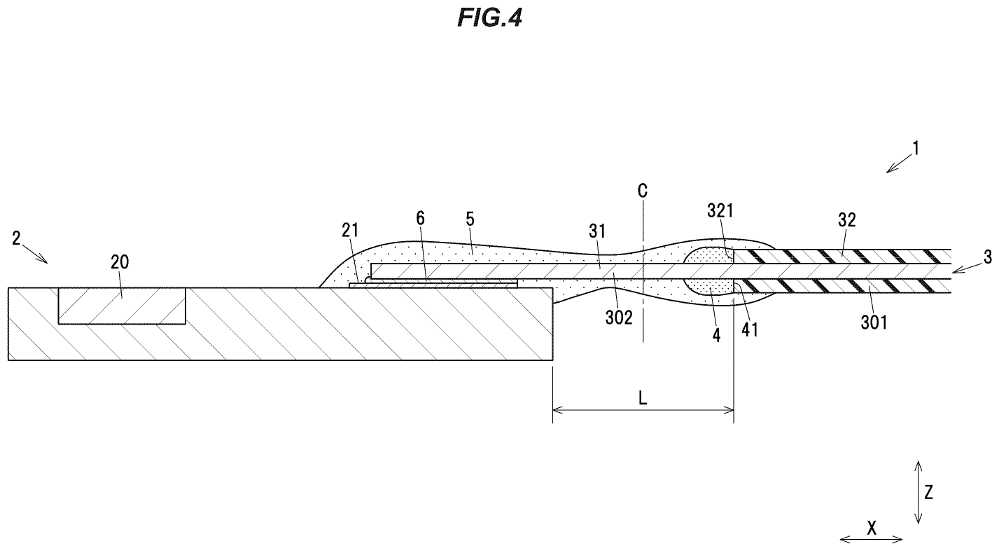

FIG. 1 is a perspective view showing an electric wire connection structure 1 in the first embodiment. FIG. 2 is a plan view showing the electric wire connection structure 1. FIG. 3 is a cross-sectional view passing through core wires 31 of plural electric wires 3 and showing the electric wire connection structure 1. FIG. 4 is a cross-sectional view taken along line IV-IV in FIG. 2 as viewed in the arrow direction.

The electric wire connection structure 1 in the first embodiment constitutes a device used for a medical device that is inserted into the body. Medical devices inserted into the body must be small in size and diameter to e.g. allow for insertion into the body and reduce the burden on the human body. As an example, a guidewire equipped with a pressure sensor is expected to be a medical device that includes the electric wire connection structure 1 in the first embodiment. The guidewire is to guide the movement of the catheter inside the body.

The use of the electric wire connection structure 1 is not limited thereto, and the electric wire connection structure 1 may be used for, e.g., a medical device inserted into the body, other than the guidewire. The wire connection structure 1 can also be used in applications other than medical devices as long as the structure is used to connect plural electric wires 3 to plural electrodes 21 of an electronic component 2.

The electric wire connection structure 1 in the first embodiment includes the electronic component 2, the plural electric wires 3, a core wire fixing member 4, and a sealing material 5. The electronic component 2 includes the plural electrodes 21. The plural electric wires 3 each include a covered portion 301 at which a core wire 31 is covered with a covering 32, and a core wire exposed portion 302 at which the core wire 31 is exposed from the covering 32. The core wire exposed portions 302 of the plural electric wires 3 are arranged in parallel. Hereinafter, the longitudinal direction of each core wire exposed portion 302 will be referred to as the “axial direction X”, the direction in which the plural core wire exposed portions 302 are aligned will be referred to as the width direction Y, and the direction orthogonal to both the axial direction X and the width direction Y will be referred to as the height direction Z. The core wire fixing member 4 fixes the plural core wire exposed portions 302 to each other. The sealing material 5 seals the plural electrodes 21, the tip portions of the plural electric wires 3, and the core wire fixing member 4. For convenience, FIG. 1 shows only the respective outlines of the core wire fixing member 4 and the sealing material 5, which are indicated by broken lines.

The electronic component 2 is formed in a rectangular parallelepiped shape that is long in the axial direction X and has a thickness in the height direction Z. The electronic component 2 includes a sensor unit 20 and the plural electrodes 21. In the first embodiment, the sensor unit 20 is a pressure sensor that detects pressure inside a blood vessel. Although detailed illustrations are omitted, the pressure sensor includes a diaphragm that flexes under the pressure of blood and a Wheatstone bridge having a strain gauge whose electrical resistance decreases with the deformation of the diaphragm (e.g., the pressure sensor described in JP 2023-39717A). The electronic component 2 may be an electronic component other than the sensor.

The plural electrodes 21 consist of a total of three electrodes: one input electrode to apply voltage to the Wheatstone bridge of the sensor unit 20, and two output electrodes respectively connected to two points of the Wheatstone bridge between which the potential difference changes according to the pressure acting on the diaphragm. The number of electrodes 21 provided on the electronic component 2 may be two or may be not less than four as long as it is more than one. The same applies to the number of electric wires 3 connected to the electrodes 21.

The three electrodes 21 are formed of a wiring pattern formed a surface of the electronic component 2 on one side in the height direction Z. The three electrodes 21 each have a rectangular plate shape which is long in the axial direction X and short in the width direction Y and are arranged in parallel in the width direction Y. The width of the three electrodes 21 is, e.g., not more than 0.1 mm. The three electrodes 21 are arranged at a pitch of, e.g., not more than 0.1 mm. The core wire exposed portions 302 of the three electric wires 3 are respectively connected to the three electrodes 21.

The electric wire 3 includes the core wire 31 and the covering 32. The core wire 31 is made of a highly conductive material such as a copper alloy plated with silver or gold, etc., and is formed of a solid wire with a circular cross-section. Plating the core wire 31 with silver or gold, etc., makes it easier to solder the core wire 31 to the electrode 21, as described later. The diameter of the core wire 31 is, e.g., not more than 0.05 mm. In this regard, the core wire 31 may be formed of a stranded wire. However, when using the solid wire, it is easier to form a straight shape, and the three core wire exposed portions 302 are kept parallel to each other easily. In case the core wire 31 is a stranded wire, the diameter of the core wire 31 can be the diameter of a circumscribed circle of the plural strands constituting the core wire 31.

The covering 32 is made of an electrically insulating resin. In the first embodiment, the covering 32 is made of, e.g., a highly heat-resistant fluorine-based resin such as PFA (tetrafluoroethylene-perfluoroalkyl vinyl ether copolymer) or PTFE (polytetrafluoroethylene).

As described above, the electric wire 3 includes the covered portion 301, at which the core wire 31 is covered with the covering 32, and the core wire exposed portion 302, at which the core wire 31 is exposed from the covering 32. Tip end surfaces 321 of the respective coverings 32 of the three electric wires 3 are aligned at the same position, and the core wire exposed portions 302 protrude from the tip end surfaces 321 of the coverings 32 toward the tip side. As shown in FIGS. 3 and 4, a length L in the axial direction X from the tip end surfaces 321 of the coverings 32 of the three electric wires 3 to the electronic component 2 is, e.g., not more than 0.5 mm. The core wire exposed portions 302 of the three electric wires 3 are arranged in a row, parallel to each other. The core wire exposed portions 302 in positions parallel to the electrode 21 are connected to the electrode 21.

The plural core wire exposed portions 302 are respectively connected to the plural electrodes 21 using a conductive bonding material 6 that melts and hardens with temperature change. In the first embodiment, the conductive bonding material 6 is made of a solder that melts on heating and hardens on cooling, particularly an Au-Sn-based solder with a relatively high melting temperature. However, the material of the conductive bonding material 6 is not limited thereto and may be a solder made of another material. The three core wire exposed portions 302 are fixed to each other by the core wire fixing member 4 at the opposite side to the portions connected to electrodes 21.

The core wire fixing member 4 fixes the three core wire exposed portions 302 such that the pitch between the three core wire exposed portions 302 is the same as the pitch between the three electrodes 21. The core wire fixing member 4 covers the entire circumference of each of the three core wire exposed portions 302 and integrates them together.

The core wire fixing member 4 is formed by hardening a molten resin filling around the three core wire exposed portions 302 and has electrical insulation properties. The core wire fixing member 4 is made of a heat-resistant material that can withstand the heating temperature when joining the core wire exposed portion 302 to the electrode 21 with the conductive bonding material 6. For example, the core wire fixing member 4 is made of an ultraviolet (UV)-curable epoxy adhesive, etc. The core wire fixing member 4 can be made of, e.g., a material not adhesive to the covering 32, but may be made of a material adhesive to the covering 32 in the same manner as the sealing material 5 (described later).

The core wire fixing member 4 covers base portions of the three core wire exposed portions 302 (i.e., the end portions on the side where the tip end surfaces 321 of the coverings 32 are located). As shown in FIGS. 3 and 4, at least a portion of the core wire fixing member 4 is arranged on the side where the tip end surfaces 321 of the coverings 32 are located, relative to a center position C between the electronic component 2 and the tip end surfaces 321 of the coverings 32 in the axial direction X. In the first embodiment, the entire core wire fixing member 4 is arranged on the side where the tip end surfaces 321 of the coverings 32 are located, relative to the center position C. In particular, in the first embodiment, the core wire fixing member 4 has a contact surface 41 which is in contact with the tip end surfaces 321 of the three coverings 32. When the positions of the tip end surfaces 321 of the three coverings 32 are not aligned, the center position C is a center position between the electronic component 2 and the tip end surface 321 located closest to the electronic component 2 among the tip end surfaces 321 of the three coverings 32.

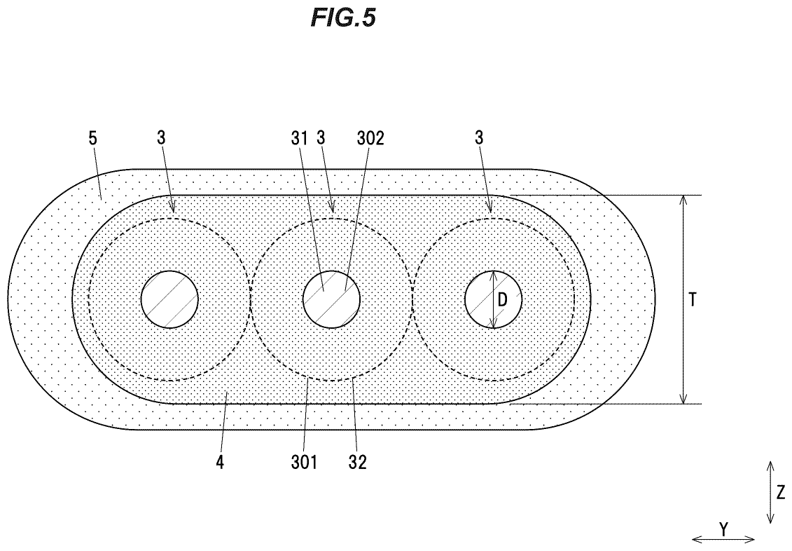

FIG. 5 is a cross-sectional view taken along line V-V in FIG. 2 as viewed in the arrow direction. The core wire fixing member 4 is formed in a bulk shape with a thickness T (i.e., the dimension in the height direction Z) of not less than a diameter D of the core wire 31. In other words, the core wire fixing member 4 is not a thin film formed on the surface of each core wire 31, but is formed in a thick shape that fills gaps between the three core wire exposed portions 302 and has the thickness T of not less than the diameter D of the core wire 31. By forming the core wire fixing member 4 in a bulk shape, the strength of the core wire fixing member 4 is ensured and the distances between the three core wire exposed portions 302 are easily maintained. When, e.g., the core wire fixing member 4 has a distorted shape and has different thicknesses in different portions, the thickness T means the maximum thickness of the core wire fixing member 4.

The sealing material 5 covers the three electrodes 21, the three core wire exposed portions 302, tip portions of the three covered portions 301 and the core wire fixing member 4 to suppress ingress of moisture such as blood into the conductive portions of the wire connection structure 1. The sealing material 5 is made of a material that is adhesive to the fluorine-based resin constituting the covering 32. As an example, the sealing material 5 can be made of a cyanoacrylate adhesive, etc. that has been subjected to treatment to improve adhesion, such as primer treatment.

### Method for Manufacturing Electric Wire Connection Structure

Next, a method for manufacturing the electric wire connection structure 1 in the first embodiment will be described.

In the method for manufacturing the electric wire connection structure 1 in the first embodiment, an electric wire preparation step, an electric wire fixing step, a connecting step and a sealing step are performed in this order.

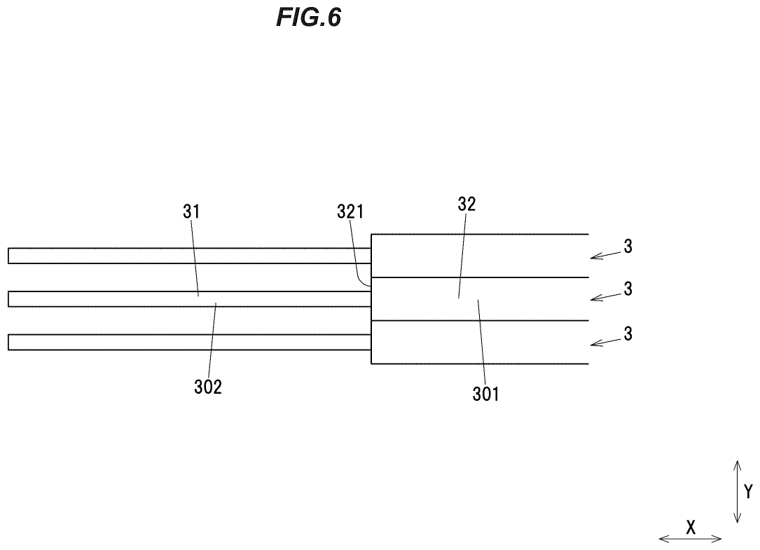

FIG. 6 is a plan view showing the three electric wires 3 prepared in the electric wire preparation step. In the electric wire preparation step, the three electric wires 3 each having the core wire exposed portion 302 are prepared. In the first embodiment, the core wire exposed portions 302 are formed by partially removing the coverings 32 from the three electric wires 3. The position of the tip end surface 321 of the covering 32 and the length of the core wire exposed portion 302 are the same for the three electric wires 3.

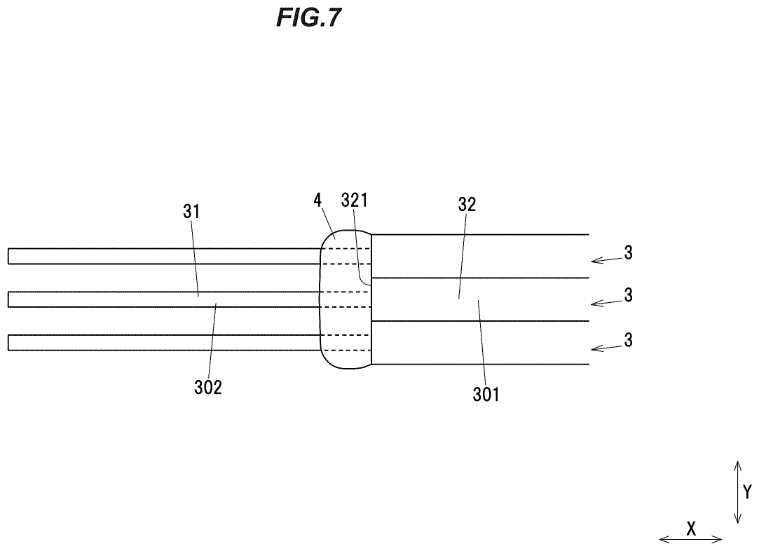

After the electric wire preparation step, the electric wire fixing step is performed. FIG. 7 is a plan view showing the three electric wires 3 and the core wire fixing member 4 after the electric wire fixing step. In the electric wire fixing step, first, the three core wire exposed portions 302 are arranged in parallel at the same pitch as the three electrodes 21. To arrange the three core wire exposed portions 302 in parallel, e.g., a tool having three grooves formed at the same pitch as the three electrodes 21 is used and the three core wire exposed portions 302 are respectively placed in the three grooves. The three core wire exposed portions 302 can thereby be arranged in parallel.

The three core wire exposed portions 302 arranged in parallel are fixed to each other by the core wire fixing member 4. When a UV-curable resin is used as the core wire fixing member 4, a molten resin to be the core wire fixing member 4 is applied to cover the three core wire exposed portions 302 and is cured by irradiation with ultraviolet light, and the core wire fixing member 4 is thereby obtained. In the first embodiment, the core wire fixing member 4 is formed so as to be in contact with the tip end surfaces 321 of the three coverings 32 as described above. In case that the core wire fixing member 4 is formed so as to be in contact with the tip end surfaces 321 of the three coverings 32, the molten resin to be the core wire fixing member 4 easily spreads along the tip end surfaces 321 of the three coverings 32 and is easily formed in a bulk shape. The core wire fixing member 4 may be formed by a method other than coating application, and, e.g., a mold, etc., may be used.

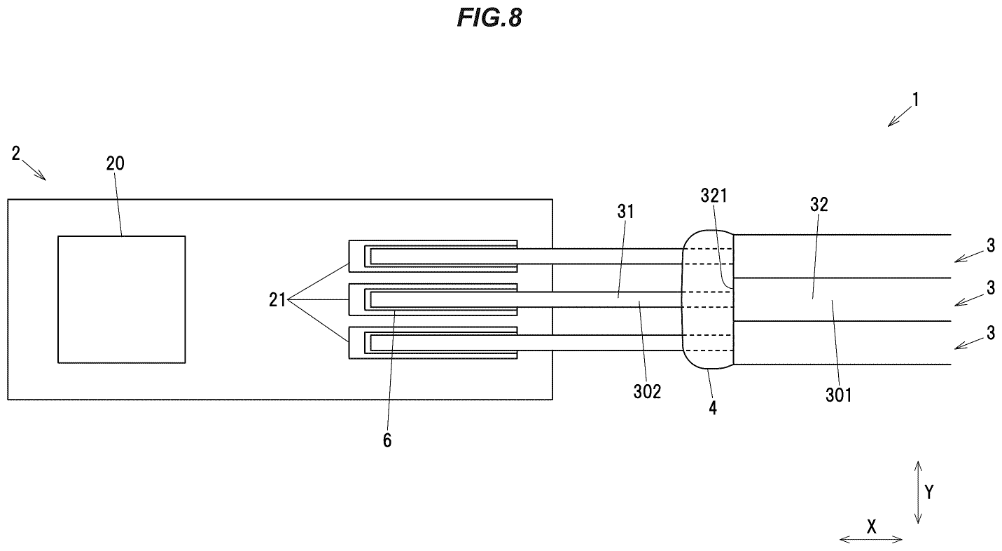

After the electric wire fixing step, the connecting step is performed. FIG. 8 is a plan view showing the electronic component 2, three conductive bonding materials 6, the three electric wires 3, and the core wire fixing member 4 after the connecting step. In the connecting step, the three core wire exposed portions 302 are connected to the three electrodes 21 using the conductive bonding materials 6. In the first embodiment, first, a solder paste to be the conductive bonding material 6 is placed on each of the three electrodes 21 of the electronic component 2 in the connecting step. Then, the three core wire exposed portions 302 are positioned to respectively face the three electrodes 21 and are brought into contact with the solder pastes respectively placed on the three electrodes 21. The solder pastes are then melted and hardened by reflowing at a temperature of about 300° C. as an example, thereby forming the conductive bonding materials 6 that mechanically and electrically connect the three core wire exposed portions 302 to the three electrodes 21. The material of the core wire fixing member 4 is selected so that the core wire fixing member 4 can withstand the high temperature environment at this time.

After the connecting step, the sealing step is performed. In the sealing step, the three electrodes 21, the three core wire exposed portions 302, the tip portions of the three covered portions 301 and the core wire fixing member 4 are covered with a molten resin to be the sealing material 5 and the molten resin is then hardened, thereby forming the sealing material 5 as shown in FIGS. 2 to 4.

The electric wire connection structure 1 in the first embodiment can be manufactured as described above.

### Functions and Effects of the First Embodiment

The electric wire connection structure 1 in the first embodiment includes the core wire fixing member 4 that fixes the plural core wire exposed portions 302 to each other. Therefore, it is easy to maintain the distances between the plural core wire exposed portions 302. This suppresses contact and conduction between the core wire exposed portions 302 due to, e.g., deformation of the core wire exposed portions 302 during the manufacturing process.

In addition, the core wire fixing member 4 is formed by hardening a molten resin filling around the plural core wire exposed portions 302 and is in contact with the tip end surfaces 321 of the plural coverings 32. Therefore, the molten resin to be the core wire fixing member 4 easily spreads along the tip end surfaces 321 of the plural coverings 32 and is easily formed in a bulk shape. The bulk shape of the core wire fixing member 4 increases the strength of the core wire fixing member 4 and suppresses separation of the core wire exposed portions 302 from the core wire fixing member 4.

In addition, the electric wire connection structure 1 in the first embodiment further includes the sealing material 5 that seals the plural electrodes 21, the tip portions of the plural electric wires 3 and the core wire fixing member 4. Therefore, e.g., ingress of moisture, etc. to the plural electrodes 21 and the plural electric wires 3 is suppressed. In addition, separately forming the core wire fixing member 4 and the sealing material 5 increases the degrees of freedom in selecting the materials thereof. For example, a material which is adhesive to the portions to be sealed (in the first embodiment, the three electrodes 21, the core wires 31 and the coverings 32 of the three electric wires 3, and the core wire fixing member 4) can be selected as the sealing material 5, while a material which does not have such adhesive properties can be used as the core wire fixing member 4.

In addition, at least a portion of the core wire fixing member 4 is arranged on the side where the tip end surfaces 321 of the coverings 32 are located, relative to the center position C between the electronic component 2 and the tip end surfaces 321 of the coverings 32 in the axial direction X. As a result, an impact (e.g., thermal or mechanical impact, etc.) on the core wire fixing member 4 at the time of connecting the electrodes 21 to the core wire exposed portions 302 can be suppressed without increasing the length L in the axial direction X from the tip end surfaces 321 of the coverings 32 of the three electric wires 3 to the electronic component 2. This reduces the sealing area of the sealing material 5, thereby reducing the overall weight of the electric wire connection structure 1 and simplifying the sealing step of forming the sealing material 5.

In addition, the coverings 32 are made of a fluorine-based resin, and the sealing material 5 seals up to the tip portions of the coverings 32 and is made of a material that is adhesive to the fluorine-based resin. Materials which can adhere to fluorine-based resins are limited. However, in the first embodiment, since ingress of moisture to, e.g., the plural electrodes 21 and the conductive portions of the plural electric wires 3 through the interface between the coverings 32 and the sealing material 5 is suppressed, the core wire fixing member 4 is not necessarily required to have adhesion to the portions to be in contact therewith, which increases the degrees of freedom in selecting the material.

In addition, the core wire fixing member 4 is formed in a bulk shape with the thickness T of not less than the diameter D of the core wire 31. This increases the strength of the core wire fixing member 4, resulting in that separation of the core wire exposed portions 302 from the core wire fixing member 4 due to cracking, etc., of the core wire fixing member 4 is suppressed.

In addition, the plural core wire exposed portions 302 are respectively connected to the plural electrodes 21 using the conductive bonding materials 6 that melt and harden with temperature change. This is a condition in which the core wire fixing member 4 is affected by thermal changes at the time of connecting the core wire exposed portions 302 to the electrodes 21 and thermal stress is likely to be generated. However, in the first embodiment, since the core wire fixing member 4 is formed in a bulk shape, separation of the core wire exposed portions 302 from the core wire fixing member 4 due to thermal stress is suppressed.

In addition, in the first embodiment, the electric wire connection structure 1 is used for a medical device that is inserted into the body. That is, each component constituting the electric wire connection structure 1 needs to be extremely small so that the medical device including the electric wire connection structure I can be inserted into the body. In such a case, it is extremely difficult to maintain the distances between the plural core wire exposed portions 302. However, the electric wire connection structure 1 in the first embodiment can be suitably used since the distances between the plural core wire exposed portions 302 can be maintained by using the core wire fixing member 4 as described above.

In addition, the method for manufacturing the electric wire connection structure 1 in the first embodiment includes the electric wire fixing step in which the core wire exposed portions 302 of the plural electric wires 3, at which the core wires 31 are exposed from the coverings 32, are arranged in parallel and fixed by the core wire fixing member 4, and the connecting step which is performed after the electric wire fixing step and in which the core wire exposed portions 302 of the plural electric wires 3 are respectively connected to the plural electrodes 21 of the electronic component 2. In this way, the core wire exposed portions 302 can be aligned with the plural electrodes 21 in the state in which the distances between the plural core wire exposed portions 302 are fixed by the core wire fixing member 4, hence, productivity of the electric wire connection structure 1 is improved.

As described above, according to the first embodiment, it is possible to provide an electric wire connection structure in which the distances between plural core wire exposed portions are easily maintained, and a method for manufacturing an electric wire connection structure.

### Second Embodiment

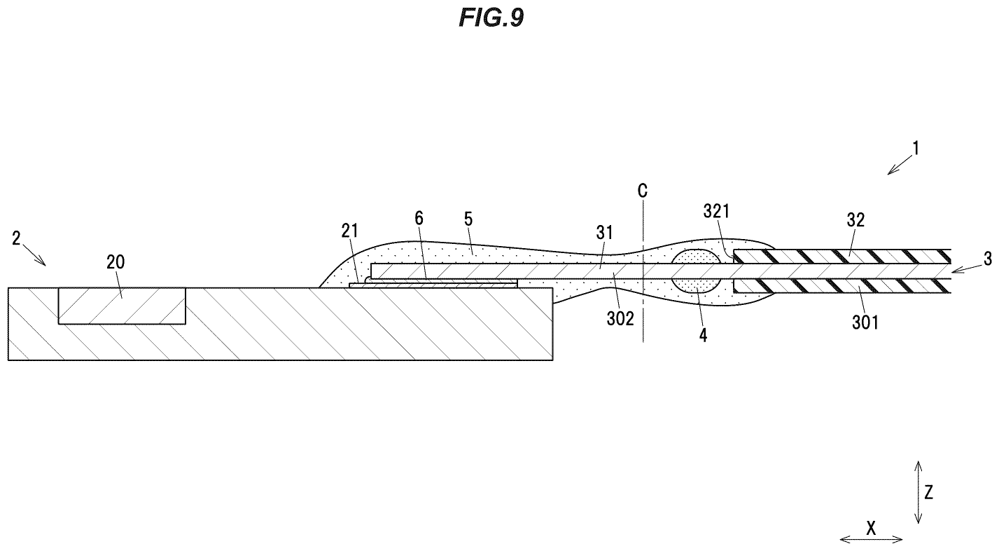

The second embodiment of the invention will be described in reference to FIG. 9. FIG. 9 is a cross-sectional view showing the electric wire connection structure 1 taken parallel to the axial direction X and the height direction Z.

In the second embodiment, the relative positions of the core wire fixing member 4 and the coverings 32 are changed from those in the first embodiment. In particular, the core wire fixing member 4 is arranged at a distance from the tip end surfaces 321 of the coverings 32 of the three electric wires 3. Also in the second embodiment, the core wire fixing member 4 is arranged on the side where the tip end surfaces 321 of the coverings 32 are located, relative to the center position C between the electronic component 2 and the tip end surfaces 321 of the coverings 32 in the axial direction X. The core wire fixing member 4 covers only the three core wire exposed portions 302. The space between the core wire fixing member 4 and the tip end surfaces 321 of the coverings 32 of the three electric wires 3 is filled with the sealing material 5.

The other configurations in the second embodiment are the same as in the first embodiment.

Among the reference signs used in the second embodiment onwards, the same reference signs as those used in the already-described embodiment indicate the same constituent elements, etc., as those in the already-described embodiment, unless otherwise specified.

### Functions and Effects of the Second Embodiment

In the second embodiment, the core wire fixing member 4 is located at a distance from the tip end surfaces 321 of the coverings 32, and the space between the core wire fixing member 4 and the tip end surfaces 321 of the coverings 32 of the plural electric wires 3 is filled with the sealing material 5. Since the sealing material 5 which is adhesive to the coverings 32 is arranged also on the tip end surfaces 321 of the coverings 32, reaching of moisture to the conductive portions of the plural electric wires 3 through between the sealing material 5 and the coverings 32 is suppressed.

The second embodiment also has the same functions and effects as those in the first embodiment.

Summary of the embodiments

Technical ideas understood from the embodiments will be described below citing the reference signs, etc., used for the embodiments. However, each reference sign, etc. described below is not intended to limit the constituent elements in the claims to the members, etc., specifically described in the embodiments.

According to the first feature, an electric wire connection structure 1 includes a plurality of electric wires 3 each including a covered portion 301, at which a core wire 31 is covered with a covering 32, and a core wire exposed portion 302, at which the core wire 31 is exposed from the covering 32, the exposed core wire portions 302 being arranged in parallel to each other; an electronic component 2 includes a plurality of electrodes 21 respectively connected to the core wire exposed portions 302 of the plurality of electric wires 3; and a core wire fixing member 4 to fix the plurality of core wire exposed portions 302 to each other.

According to the second feature, in the electric wire connection structure 1 as described by the first feature, the core wire fixing member 4 is formed by hardening a molten resin filling around the plurality of core wire exposed portions 302 and is in contact with a tip end surface 321 of the covering 32 of at least one of the plurality of electric wires 3.

According to the third feature, the electric wire connection structure 1 as described by the first or second feature further includes a sealing material 5 to seal the plurality of electrodes 21, tip portions of the plurality of electric wires 3, and the core wire fixing member 4.

According to the fourth feature, in the electric wire connection structure 1 as described by the third feature, at least a portion of the core wire fixing member 4 is arranged on the tip end surface 321 side relative to a center position C between the electronic component 2 and the tip end surface 321 of the covering 32 in a longitudinal direction X of each of the core wire exposed portions 302.

According to the fifth feature, in the electric wire connection structure 1 as described by the third or fourth feature, the covering 32 includes a fluorine-based resin, and the sealing material 5 seals up to tip portions of the coverings 32 of the plurality of electric wires 3 and includes a material that is adhesive to the fluorine-based resin.

According to the sixth feature, in the electric wire connection structure 1 as described by the fifth feature, the core wire fixing member 4 is located at a distance from the tip end surfaces 321 of the coverings 32 of the plurality of electric wires 3, and a space between the core wire fixing member 4 and the tip end surfaces 321 of the coverings 32 of the plurality of electric wires 3 is filled with the sealing material 5.

According to the seventh feature, in the electric wire connection structure 1 as described by any one of the first to sixth features, the core wire fixing member 4 is formed in a bulk shape with a thickness T of not less than a diameter D of the core wire 31.

According to the eighth feature, in the electric wire connection structure 1 as described by the seventh feature, the plurality of core wire exposed portions 302 are respectively connected to the plurality of electrodes 21 using a conductive bonding material 6 that melts and hardens with temperature change.

According to the ninth feature, the electric wire connection structure 1 as described by any one of the first to eighth features is used for a medical device that is inserted into the body.

According to the tenth feature, a method for manufacturing an electric wire connection structure 1 includes arranging in parallel core wire exposed portions 302 of a plurality of electric wires 3 at which core wires 31 are exposed from coverings 32, and fixing the core wire exposed portions 302 with a core wire fixing member 4, thereby fixing electric wires; and after the fixing electric wires, connecting the core wire exposed portions 302 of the plurality of electric wires 3 respectively to a plurality of electrodes 21 of an electronic component 2.

Although the embodiments of the invention have been described, the invention according to claims is not to be limited to the embodiments described above. Further, please note that not all combinations of the features described in the embodiments are necessary to solve the problem of the invention. In addition, the invention can be appropriately modified and implemented without departing from the gist thereof.

1. An electric wire connection structure, comprising:
a plurality of electric wires each comprising a covered portion, at which a core wire is covered with a covering, and a core wire exposed portion, at which the core wire is exposed from the covering, the exposed core wire portions being arranged in parallel to each other;
an electronic component comprising a plurality of electrodes respectively connected to the core wire exposed portions of the plurality of electric wires; and
a core wire fixing member to fix the plurality of core wire exposed portions to each other.

2. The electric wire connection structure, according to claim 1, wherein the core wire fixing member is formed by hardening a molten resin filling around the plurality of core wire exposed portions and is in contact with a tip end surface of the covering of at least one of the plurality of electric wires.

3. The electric wire connection structure, according to claim 1, further comprising:
a sealing material to seal the plurality of electrodes, tip portions of the plurality of electric wires, and the core wire fixing member.

4. The electric wire connection structure, according to claim 3, wherein at least a portion of the core wire fixing member is arranged on a tip end surface-side relative to a center position between the electronic component and the tip end surface of the covering in a longitudinal direction of each of the core wire exposed portions.

5. The electric wire connection structure, according to claim 3, wherein the covering comprises a fluorine-based resin, and wherein the sealing material seals up to tip portions of the coverings of the plurality of electric wires and comprises a material that is adhesive to the fluorine-based resin.

6. The electric wire connection structure, according to claim 5, wherein the core wire fixing member is located at a distance from the tip end surfaces of the coverings of the plurality of electric wires, and wherein a space between the core wire fixing member and the tip end surfaces of the coverings of the plurality of electric wires is filled with the sealing material.

7. The electric wire connection structure, according to claim 1, wherein the core wire fixing member is formed in a bulk shape with a thickness of not less than a diameter of the core wire.

8. The electric wire connection structure, according to claim 7, wherein the plurality of core wire exposed portions are respectively connected to the plurality of electrodes using a conductive bonding material that melts and hardens with temperature change.

9. The electric wire connection structure, according to claim 1, for a medical device configured to be inserted into a body.

10. A method for manufacturing an electric wire connection structure, comprising:
arranging in parallel core wire exposed portions of a plurality of electric wires at which core wires are exposed from coverings, and fixing the core wire exposed portions with a core wire fixing member, thereby fixing electric wires; and
after the fixing electric wires, connecting the core wire exposed portions of the plurality of electric wires respectively to a plurality of electrodes of an electronic component.
